(19) United States
(12) Patent Application Publication
(10) Pub. No.: US 2026/0103222 A1
(43) Pub. Date: Apr. 16, 2026

(54) VEHICLE AND VEHICLE ACCIDENT ANALYSIS SERVER AND METHOD

(71) Applicants: Hyundai Motor Company, Seoul (KR); Kia Corporation, Seoul (KR)

(72) Inventor: Sang Min LEE, Hwaseong-Si (KR)

(21) Appl. No.: 19/359,183

(22) Filed: Oct. 15, 2025

(30) Foreign Application Priority Data
Oct. 16, 2024 (KR) 10-2024-0141133

(51) Int. Cl. B60W 60/00; A61B 5/00; B60R 22/48; B60W 40/08; B60W 40/105; B60W 50/00; G01P 13/00; G07C 5/00; G07C 5/04

(52) U.S. Cl. CPC B60W 60/0016; A61B 5/6893; B60R 22/48; B60W 40/08; B60W 40/105; B60W 50/0098; G01P 13/00; G07C 5/008; G07C 5/04; B60R 2022/4866; B60W 2050/0022; B60W 2050/0026; B60W 2510/30; B60W 2520/105; B60W 2530/10; B60W 2540/049; B60W 2540/221; B60W 2540/227; B60W 2556/10; B60W 2556/45

(57) ABSTRACT

A vehicle and a vehicle accident analysis server and method are disclosed. A vehicle includes a power-net domain controller (PDC) that detects information on a passenger aboard the vehicle, a gyro sensor that detects an X-axis value and a Y-axis value of the vehicle, a speed sensor that detects a speed of the vehicle, an airbag control unit (ACU) that, when an airbag deployment signal is generated by a collision, collects an X-axis value and a Y-axis value of the vehicle and vehicle speeds periodically detected for a set time from a time when the airbag deployment signal is generated, and a data connectivity unit (DCU) that provides an eCall function for collecting the passenger information, the X-axis value and Y-axis value of the vehicle, and the periodically detected vehicle speeds and transmitting the collected passenger information, X-axis value and Y-axis value, and periodically detected vehicle speeds to a server.

### CROSS-REFERENCE TO RELATED APPLICATION

This application claims priority to and the benefit of Korean Patent Application No. 10-2024-0141133, filed on Oct. 16, 2024, the disclosure of which is incorporated herein by reference in its entirety.

### FIELD

The present disclosure relates to a vehicle and a vehicle accident analysis server and method, and, more particularly, to a vehicle and a vehicle accident analysis server and method capable of estimating a collision severity index (CSI) and injury severity of each passenger from information related to a vehicle accident.

### BACKGROUND

Human casualties due to a car accident may be caused by the amount of impact of the accident, and information on passenger conditions collected after the occurrence of an accident can play a vital role in passenger rescue and treatment. Accordingly, some countries have legislated the installation of an eCall system to protect a driver's life in the event of an accident and collect information on the accident to share the collected information with an emergency rescue center.

However, the existing eCall system does not provide information on a point where a collision has occurred in a vehicle, and therefore, a server providing eCall services can have difficulty estimating the injury severity of passengers in the vehicle.

### SUMMARY

The present disclosure relates to providing a vehicle and a vehicle accident analysis server and method capable of determining a collision severity index of a vehicle and estimating injury severity of each passenger by using information on a point where a collision has occurred in the vehicle.

According to an aspect of the present disclosure, there is provided a vehicle accident analysis server including: one or more processors; and a memory configured to store one or more programs executed by the one or more processors, in which the processor determines an accident risk level of an accident vehicle based on information related to an accident of the accident vehicle (hereinafter referred to as “vehicle accident information”), and estimates injury severity of each passenger in the accident vehicle based on the determined accident risk level.

The processor may include: a collision severity index (CSI) calculation unit that calculates a CSI based on a plurality of pieces of first information among the vehicle accident information; a CSI correction unit that corrects the calculated CSI based on a plurality of pieces of second information among the vehicle accident information; and/or a risk level determination unit that determines the accident risk level of the accident vehicle based on the corrected CSI.

The plurality of pieces of first information among the vehicle accident information may include a weight of the accident vehicle, a vehicle speed change amount, and/or a vehicle speed at a time when a set time has elapsed from a time when the accident occurs.

The vehicle accident analysis server may further include a communication interface unit that receives a vehicle identification number from the accident vehicle and vehicle speeds periodically measured for the set time from the time when the accident occurs. The processor may further include an information generation unit that confirms a vehicle weight mapped to the received vehicle identification number in a database (DB) to use the confirmed vehicle weight as the weight of the accident vehicle, and/or calculates the vehicle speed change amount from the periodically measured vehicle speeds.

The plurality of pieces of second information may include an airbag deployment signal indicating that an airbag in the accident vehicle has been deployed, and the CSI correction unit may reduce the calculated CSI using a coefficient mapped to the airbag deployment signal to perform a primary correction.

The plurality of pieces of second information may further include a vehicle collision angle calculated from a gyro sensing value of the accident vehicle, and the CSI correction unit may correct the primarily corrected CSI using a coefficient mapped to the calculated vehicle collision angle.

The accident vehicle may transmit an X-axis value and a Y-axis value of a gyro sensor detected at a time when the airbag deployment signal is input, and the processor may further include an information generation unit that calculates the vehicle collision angle based on the X-axis value and Y-axis value of the gyro sensor.

The CSI correction unit may classify a collision type of the accident vehicle as one of frontal collision, oblique collision, rear-end collision, and lateral collision based on the calculated vehicle collision angle, and correct the primarily corrected CSI using a coefficient mapped to the classified collision type.

The processor may further include a passenger accident estimation unit that estimates the injury severity of each passenger based on the accident risk level determined by the risk level determination unit and a plurality of pieces of third information among the vehicle accident information.

The plurality of pieces of third information may be information on passengers aboard the accident vehicle, and include the number of passengers, the passengers' boarding seat locations, and whether the passengers are wearing seat belts, and the passenger accident estimation unit may calculate the injury severity of each passenger using a weight set for each passenger seat location and the determined accident risk level.

The passenger accident estimation unit may correct the injury severity calculated for each passenger using whether each passenger is wearing a seat belt and determine the corrected result as final injury severity of each passenger.

According to another aspect of the present disclosure, there may be provided a vehicle accident analysis method of a server, which may include a processor and a memory configured to store one or more programs executed by the processor, including: determining an accident risk level of an accident vehicle based on information related to an accident of the accident vehicle (hereinafter referred to as “vehicle accident information”); and/or estimating injury severity of each passenger in the accident vehicle based on the determined accident risk level.

The determining of the accident risk level may include: calculating a CSI based on a plurality of pieces of first information among the vehicle accident information; correcting the calculated CSI based on a plurality of pieces of second information among the vehicle accident information; and determining the accident risk level of the accident vehicle based on the corrected CSI.

The plurality of pieces of first information among the vehicle accident information may include a weight of the accident vehicle, a vehicle speed change amount, and/or a vehicle speed at a time when a set time has elapsed from a time when the accident occurs.

The vehicle accident analysis method may further include, prior to the calculating of the CSI, confirming the vehicle weight mapped to the vehicle identification number in a DB to use the confirmed vehicle weight as the weight of the accident vehicle and calculating the vehicle speed change amount from periodically measured vehicle speeds, in which the accident vehicle may transmit a vehicle identification number and the vehicle speeds periodically measured for the set time from the time when the accident occurs.

The plurality of pieces of second information may include an airbag deployment signal indicating that an airbag in the accident vehicle has been deployed, and, in the correcting of the CSI, the calculated CSI may be reduced using a coefficient mapped to the airbag deployment signal to perform a primary correction.

The plurality of pieces of second information may further include a vehicle collision angle calculated from a gyro sensing value of the accident vehicle, and in the correcting of the CSI, the primarily corrected CSI may be corrected using a coefficient mapped to the calculated vehicle collision angle.

The vehicle accident analysis method may further include, prior to the correcting the calculated CSI, calculating the vehicle collision angle based on an X-axis value and a Y-axis value of the gyro sensor, in which the accident vehicle may transmit the X-axis value and Y-axis value of a gyro sensor detected at a time when the airbag deployment signal is input.

In the correcting of the calculated CSI, a collision type of the accident vehicle may be classified as one or more of frontal collision, oblique collision, rear-end collision, and/or lateral collision based on the calculated vehicle collision angle, and the primarily corrected CSI may be corrected using a coefficient mapped to the classified collision type.

In the estimating of the injury severity, the injury severity of each passenger may be estimated based on the accident risk level determined in the determining of the risk level and/or a plurality of pieces of third information among the vehicle accident information.

The plurality of pieces of third information may be information on passengers aboard the accident vehicle, and include the number of passengers, the passengers' boarding seat locations, and/or whether the passengers are wearing seat belts, and in the estimating of the injury severity, the injury severity of each passenger may be calculated using a weight set for each passenger seat location and the determined accident risk level.

In the estimating of the injury severity, the injury severity calculated for each passenger may be corrected using whether each passenger is wearing a seat belt, and the corrected result may be determined as final injury severity of each passenger.

According to still another aspect of the present disclosure, there is provided a vehicle including: a power-net domain controller (PDC) that detects information on a passenger aboard the vehicle; a gyro sensor that detects an X-axis value and a Y-axis value of the vehicle; a speed sensor that detects a speed of the vehicle; an ACU that, when an airbag deployment signal is generated by a collision, collects an X-axis value and a Y-axis value of the vehicle and vehicle speeds periodically detected for a set time from a time when the airbag deployment signal is generated; and a DCU that, when the airbag deployment signal is generated, provides an eCall function for collecting the passenger information, the X-axis value and Y-axis value of the vehicle, and the periodically detected vehicle speeds and transmitting the collected passenger information, X-axis value and Y-axis value, and periodically detected vehicle speeds to a vehicle accident analysis server.

As another example, a computing device may be located in a vehicle and may be configured to monitor and report accidents associated with the vehicle. The computing device may detect, via one or more sensors, an occurrence of an accident associated with the vehicle. The computing device may then calculate, based on data from a gyro of the vehicle, a vehicle collision angle and calculate, based on the vehicle collision angle and one or more properties of the vehicle, a collision severity index corresponding to the accident. Then, the computing device may obtain, based on seat belt sensors of the vehicle, passenger data corresponding to one or more passengers in the vehicle at a time of the accident and estimate, based on the collision severity index, an injury level for each of the one or more passengers. The computing device may then transmit, to a remote server, the estimated injury level for each of the one or more passengers.

The features briefly summarized above with respect to the present disclosure are merely exemplary aspects of the detailed description of the disclosure to be described below, and do not limit the scope of the disclosure.

### BRIEF DESCRIPTION OF DRAWINGS

The above and other objects, features and advantages of the present disclosure will become more apparent to those of ordinary skill in the art by describing exemplary examples thereof in detail with reference to the accompanying drawings, in which:

FIG. 1 is a diagram illustrating a vehicle communicating with another device to transmit and receive data;

FIG. 2 is a diagram illustrating a module constituting a vehicle;

FIG. 3 is a diagram illustrating a vehicle accident analysis system;

FIG. 4 is a block diagram illustrating a vehicle;

FIG. 5 is a diagram illustrating an X-axis, a Y-axis, and a Z-axis of a vehicle;

FIG. 6 is a block diagram illustrating a vehicle accident analysis server;

FIG. 7 is a block diagram illustrating a configuration of a processor;

FIG. 8 is a diagram illustrating an example of initial accident information input to the vehicle accident analysis server and an output value generated from the initial accident information;

FIG. 9 is a flowchart illustrating a method of providing initial accident information of a vehicle; and

FIG. 10 is a flowchart illustrating a vehicle accident analysis method.

### DETAILED DESCRIPTION

Hereinafter, examples of the present disclosure will be described in detail with reference to the accompanying drawings so that those skilled in the art to which the present disclosure pertains may easily practice the present disclosure. However, the present disclosure may be modified in various different forms and is not limited to examples described herein.

Further, in describing exemplary examples of the present disclosure, well-known functions or constructions will not be described in detail since they may unnecessarily obscure the understanding of the present disclosure. In the drawings, parts not related to the description of the present disclosure are omitted, and similar reference numerals are attached to similar parts.

In the present disclosure, when a component is said to be “connected,” “coupled,” or “joined” to another component, this may include not only a direct connection relationship, but also an indirect connection relationship where another component exists therebetween. In addition, when a component “includes” or “has” another component, this means that the component may further include other components, not excluding the inclusion of the other components unless otherwise stated.

In the present disclosure, terms such as “first” and “second” are used only for the purpose of distinguishing one component from other components, and do not limit the order, importance, or the like of components unless otherwise specified. Accordingly, within the scope of the present disclosure, a first component in an example may be referred to as a second component in another example, and similarly, a second component in an example may be referred to as a first component in other examples.

In the present disclosure, components distinguished from each other are intended to clearly explain each feature, and do not mean that the components are necessarily separated. That is, a plurality of components may be integrated to be formed in a single hardware or software unit, or a single component may be distributed to be formed in a plurality of hardware or software units. Accordingly, even when not described separately, such integrated or distributed examples are also included in the scope of the present disclosure.

In the present disclosure, components described in various examples are not necessarily essential components, and some of the components may be optional components. Therefore, examples composed of a subset of components described in an example are also included in the scope of the present disclosure. In addition, examples including other components in addition to the components described in various examples are also included in the scope of the present disclosure.

In the present disclosure, phrases such as “A or B,” “at least one of A and B,” “at least one of A or B,” “A, B, or C,” “at least one of A, B and C,” and “at least one of A, B, or C” or a combination thereof may include any one of the items listed together in the corresponding phrase, or all possible combinations thereof.

Various advantages and features of the present disclosure and methods accomplishing the same will become apparent from the following detailed description of examples with reference to the accompanying drawings. However, the present disclosure is not limited to exemplary examples disclosed below but may be implemented in various different forms. These examples will be provided only in order to make the disclosure of the present disclosure complete and allow those skilled in the art to which the present disclosure pertains to completely recognize the scope of the present disclosure.

In addition, in this specification, terms such as “module,” “unit,” “device,” and “server” may be intended to refer to the functional and structural combination of hardware and software driven by or for driving the hardware. For example, the “module” or “unit” may be realized as a processor and a memory. The “processor” should be widely construed to include a general-purpose processor, a central processing unit (CPU), a microprocessor, a digital signal processor (DSP), a microcontroller, a state machine, or the like. In some environments, the “processor” may refer to an application-specific integrated circuit (ASIC), a programmable logic device (PLD), or a field-programmable gate array (FPGA), and the like. For example, the “processor” may refer to a combination of processing devices such as a combination of a DSP and a microprocessor, a combination of a plurality of microprocessors, a combination of one or more microprocessors combined with a DSP core, or any other such combination. Moreover, the “memory” should be widely construed to include any electronic component capable of storing electronic information. The “memory” may refer to various types of processor-readable medium such as a random-access memory (RAM), a read only memory (ROM), a non-volatile random-access memory (NVRAM), a programmable read only memory (PROM), an erasable programmable read only memory (EPROM), an electrically erasable programmable read only memory (EEPROM), a flash memory, a magnetic or optical data storage device, and registers. When the processor can read information from a memory and/or record the information in the memory, the memory may be in a state of electronic communication with a processor. Memory integrated into a processor is in a state of electronic communication with the processor.

The one or more features described herein may be provided as a computer program stored in a computer-readable recording medium in order to be executed on a computer. The medium may either continuously store a computer-executable program or temporarily store the program for execution or download. Furthermore, the medium may be a variety of recording or storage means in the form of a single hardware device or multiple combined hardware devices, and is not limited to media directly connected to some computer system but may also be distributed across a network. Examples of such media include magnetic media such as a hard disk, a floppy disk, or a magnetic tape, optical recording media such as a CD-ROM or a DVD, magneto-optical media such as a floptical disk, and a ROM, RAM, or flash memory, among others, configured to store program instructions. Additional examples of such media include media or storage media that are managed by an app store that distributes applications or by various other sites or servers that provide or distribute software.

In a hardware implementation, processing units used for performing the techniques may be implemented within one or more ASICs, DSPs, digital signal processing devices, programmable logic devices, field-programmable gate arrays, processors, controllers, microcontrollers, microprocessors, electronic devices, or computers or combinations thereof designed to perform the functions described in the present disclosure.

An automation level of an autonomous driving vehicle may be classified as follows, according to the American Society of Automotive Engineers (SAE). At autonomous driving level 0, the SAE classification standard may correspond to “no automation,” in which an autonomous driving system is temporarily involved in emergency situations (e.g., automatic emergency braking) and/or provides warnings only (e.g., blind spot warning, lane departure warning, etc.), and a driver is expected to operate the vehicle. At autonomous driving level 1, the SAE classification standard may correspond to “driver assistance,” in which the system performs some driving functions (e.g., steering, acceleration, brake, lane centering, adaptive cruise control, etc.) while the driver operates the vehicle in a normal operation section, and the driver is expected to determine an operation state and/or timing of the system, perform other driving functions, and cope with (e.g., resolve) emergency situations. At autonomous driving level 2, the SAE classification standard may correspond to “partial automation,” in which the system performs steering, acceleration, and/or braking under the supervision of the driver, and the driver is expected to determine an operation state and/or timing of the system, perform other driving functions, and cope with (e.g., resolve) emergency situations. At autonomous driving level 3, the SAE classification standard may correspond to “conditional automation,” in which the system drives the vehicle (e.g., performs driving functions such as steering, acceleration, and/or braking) under limited conditions but transfer driving control to the driver when the required conditions are not met, and the driver is expected to determine an operation state and/or timing of the system, and take over control in emergency situations but do not otherwise operate the vehicle (e.g., steer, accelerate, and/or brake). At autonomous driving level 4, the SAE classification standard may correspond to “high automation,” in which the system performs all driving functions, and the driver is expected to take control of the vehicle only in emergency situations. At autonomous driving level 5, the SAE classification standard may correspond to “full automation,” in which the system performs full driving functions without any aid from the driver including in emergency situations, and the driver is not expected to perform any driving functions other than determining the operating state of the system. Although the present disclosure may apply the SAE classification standard for autonomous driving classification, other classification methods and/or algorithms may be used in one or more configurations described herein.

One or more features associated with autonomous driving control may be activated based on configured autonomous driving control setting(s) (e.g., based on at least one of: an autonomous driving classification, a selection of an autonomous driving level for a vehicle, etc.). Based on one or more features described herein, an operation of the vehicle may be controlled. The vehicle control may include various operational controls associated with the vehicle (e.g., autonomous driving control, sensor control, braking control, braking time control, acceleration control, acceleration change rate control, alarm timing control, forward collision warning time control, etc.), for example, based on one or more features (e.g., estimated injury severity, etc.) described herein. In an example, a computing device (e.g., a vehicle accident analysis server, the vehicle, etc.) may transmit, based on the estimated injury severity, a control signal to the accident vehicle to cause the accident vehicle to adjust at least one parameter for autonomous driving control of the accident vehicle. The computing device may be a part of a vehicle communicating with another vehicle via direct wireless communication (e.g., V2V, V2X communication) or via a server (e.g., via a mobile communication network). The computing device may be one or more servers of an accident management system (e.g., a vehicle accident analysis server).

One or more auxiliary devices (e.g., engine brake, exhaust brake, hydraulic retarder, electric retarder, regenerative brake, etc.) may also be controlled, for example, based on one or more features described herein.

One or more communication devices (e.g., a modem, a network adapter, a radio transceiver, an antenna, etc., that is capable of communicating via one or more wired or wireless communication protocols, such as Ethernet, Wi-Fi, near-field communication (NFC), Bluetooth, Long-Term Evolution (LTE), 5G New Radio (NR), vehicle-to-everything (V2X), etc.) may also be controlled, for example, based on one or more features of the extracted portion(s) of the DSSAD data, EDR data, etc.) described herein.

Minimum risk maneuver (MRM) operation(s) may also be controlled, for example, based on one or more features described herein. A minimal risk maneuvering operation (e.g., a minimal risk maneuver, a minimum risk maneuver) may be a maneuvering operation of a vehicle to minimize (e.g., reduce) a risk of collision with surrounding vehicles in order to reach a lowered (e.g., minimum) risk state. A minimal risk maneuver may be an operation that may be activated during autonomous driving of the vehicle when a driver is unable to respond to a request to intervene. During the minimal risk maneuver, one or more processors of the vehicle may control a driving operation of the vehicle for a set period of time.

Biased driving operation(s) may also be controlled, for example, based on one or more features described herein. A driving control apparatus may perform a biased driving control. To perform a biased driving, the driving control apparatus may control the vehicle to drive in a lane by maintaining a lateral distance between the position of the center of the vehicle and the center of the lane. For example, the driving control apparatus may control the vehicle to stay in the lane but not in the center of the lane. The driving control apparatus may identify or determine a biased target lateral distance for biased driving control. For example, a biased target lateral distance may comprise an intentionally adjusted lateral distance that a vehicle may aim to maintain from a reference point, such as the center of a lane or another vehicle, during maneuvers such as lane changes. This adjustment may be made to improve the vehicle's stability, safety, and/or performance under varying driving conditions, etc. For example, during a lane change, the driving control system may bias the lateral distance to keep a safer gap from adjacent vehicles, considering factors such as the vehicle's speed, road conditions, and/or the presence of obstacles, etc.

One or more sensors (e.g., IMU sensors, camera, LIDAR, RADAR, blind spot monitoring sensor, line departure warning sensor, parking sensor, light sensor, rain sensor, traction control sensor, anti-lock braking system sensor, tire pressure monitoring sensor, seatbelt sensor, airbag sensor, fuel sensor, emission sensor, throttle position sensor, inverter, converter, motor controller, power distribution unit, high-voltage wiring and connectors, auxiliary power modules, charging interface, etc.) may also be controlled, for example, based on one or more features described herein. An operation control for autonomous driving of the vehicle may include various driving control of the vehicle by the vehicle control device (e.g., acceleration, deceleration, steering control, gear shifting control, braking system control, traction control, stability control, cruise control, lane keeping assist control, collision avoidance system control, emergency brake assistance control, traffic sign recognition control, adaptive headlight control, etc.).

An autonomous driving level and/or autonomous driving activation/deactivation may also be controlled, for example, based on one or more features described herein. A driving control apparatus may perform an autonomous driving level control (e.g., a change of an autonomous driving level, a change of a required user attentiveness, etc.) or cause deactivation of an autonomous driving operation. For example, by changing the required user attentiveness, the driver may be required to place his/her hands on the driving wheel more often (e.g., at least once in a threshold time period, such as five second, 30 seconds, 1 minute, etc.). By changing the required user attentiveness, the driver may be required to look ahead more often (e.g., at least once in a threshold time period, such as five second, 30 seconds, 1 minute, etc.). By changing the autonomous driving level, one or more video contents might not be displayed on a display of the vehicle.

In the present disclosure, a “system” may include one or more computing devices and may be provided in a local or cloud form but is not limited thereto.

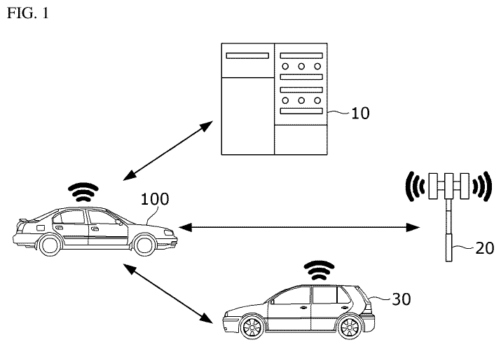

FIG. 1 is a diagram illustrating a vehicle 100 communicating with another device to transmit and receive data.

Referring to FIG. 1, the vehicle 100 may be driven autonomously or manually, and autonomous driving may be classified into semi-autonomous driving or fully autonomous driving. The fully autonomous driving is driving in which a processor 122 of the vehicle 100 completely controls control rights without user intervention. The semi-autonomous driving is driving in which a driver intervenes depending on the driving situation.

The vehicle 100 may perform wired or wireless communication with one or more servers 10, one or more intelligent transportation system (ITS) devices 20, other vehicles 30, or various types of user devices.

One or more servers 10 may provide various services related to the vehicle 100, such as various function control, status management, driving assistance, connected car service (CCS) provision, and/or traffic accident analysis service of the vehicle 100. One or more servers 10 may transmit various types of information and software modules, which are used for controlling the vehicle 100, to the vehicle 100 in response to requests and data transmitted from the vehicle 100 or a user device, for example, to support the autonomous driving or various services of the vehicle 100.

The ITS device 20 may be, for example, a road side unit (RSU) for receiving the information from the ITS, and may exchange vehicle recognition data, driving control and status data, environmental data around the vehicle, map data, etc., with the vehicle 100 through vehicle to infrastructure (V2I) to assist the user with driving or support autonomous driving of the vehicle 100.

In addition, the vehicle 100 may exchange the data listed above with other vehicles 30 through vehicle to vehicle (V2V) to support the manual driving or autonomous driving.

The vehicle 100 may perform communication with other devices 10 and 20 or other vehicles 30 based on cellular communication, wireless access in vehicular environment (WAVE) communication, dedicated short range communication (DSRC), short-range communication, or other communication methods. For example, the vehicle 100 may use a cellular communication network such as Long Term Evolution (LTE) or 5th generation (5G), a wireless fidelity (WiFi) communication network, or a WAVE communication network for communication with the server 10, the ITS device 20, and other vehicles 30. As another example, the DSRC used in the vehicle 100 may be used for communication with other vehicles 100. The communication method among the vehicle 100, the server 10, the ITS device 20, other vehicles 30, and the user devices is not limited to the above-described example.

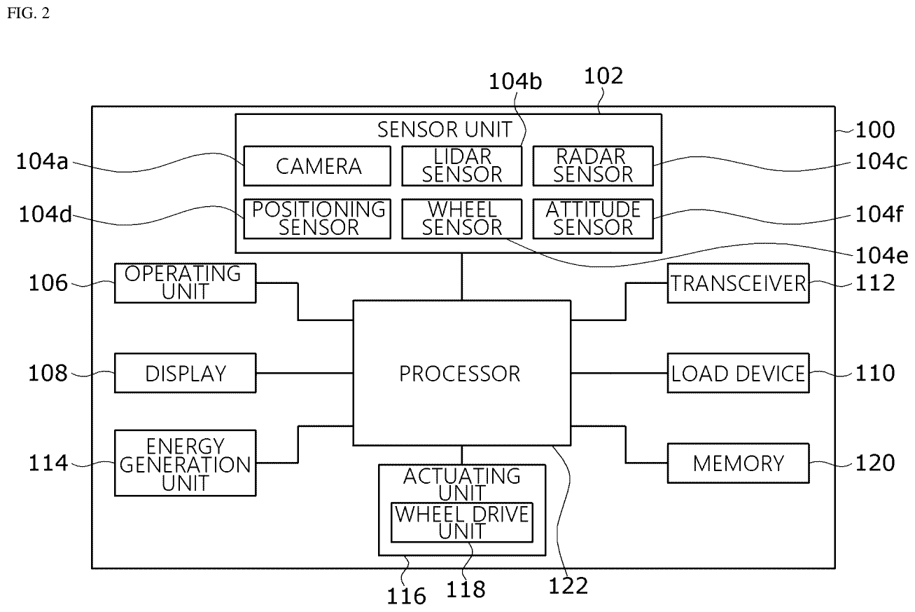

FIG. 2 is a diagram illustrating a module constituting a vehicle according to an example of the present disclosure.

The vehicle 100 may include a sensor unit 102, an operating unit 106, a display 108, a load device 110, a transceiver 112, an energy generation unit 114, an actuating unit 116, a memory 120, and a processor 122.

The sensor unit 102 may include various types of detectors for detecting various states and situations that occur in an external environment, an internal system, a user operation, and a boarding space of the vehicle 100.

Specifically, the sensor unit 102 may include at least one of an outward-facing camera 104a, a lidar sensor 104b, and a radar sensor 104c to recognize dynamic and static objects inside and outside the vehicle 100. For example, the camera 104a may capture the inside and outside of the vehicle 100 to generate image data and transmit the generated image data to the processor 122. The lidar sensor 104b may be used to generate three-dimensional spatial information that identifies a shape of an external object. The radar sensor 104c may be used to determine the existence of the external object and relative distance, speed, direction, etc.

In addition, the sensor unit 102 may further include at least one of a positioning sensor 104d, a wheel sensor 104e, and/or an attitude sensor 104f to confirm its own position, speed, driving attitude, etc. The attitude sensor 104f may include a gyro sensor, an angular velocity sensor, an acceleration sensor, etc.

The operating unit 106 may be provided as a module that a user operates for driving. For example, the operating unit 106 may be a steering wheel for manual driving, an automatic or manual transmission, an accelerator pedal, a brake pedal, etc. In addition, the operating unit 106 may be provided as a hard type interface or a touchable soft type interface on the display 108 to receive various requests related to autonomous driving (e.g., use, release, and selection of detailed functions of an autonomous driving mode) from a user.

The display 108 may be provided as a touch screen and may transmit the user's request to the processor 122. In addition, the display 108 may allow the processor 122 to display an operation status, a control status, traffic information, remaining energy information, and content requested by the driver of the vehicle 100, etc.

A load device 110 may be a type of non-driving electrical device. The load device 110 may be any of various devices such as an air conditioning system, a lighting system, a seat system, and a cooling/heating system. The cooling/heating system may cool or heat a specific part of the vehicle 100, such as a battery, a fuel cell, an internal combustion engine, and an air conditioning system.

The transceiver 112 may support mutual communication with the server 10, the ITS device 20, the nearby vehicles 30, etc. The transceiver 112 may include a module for processing, for example, the cellular communication, the WAVE, the DSRC communication, etc. In the present disclosure, the transceiver 112 may transmit data generated or stored during driving to the server 10 and receive data and software modules transmitted from the server 10. The transceiver 112 may also support communication with electronic devices carried by passengers inside the vehicle 100.

The energy generation unit 114 may generate and supply power and electricity used in a driving power system and a non-driving power system. The non-driving power system may include, but is not limited to, the sensor unit 102, the operating unit 106, the display 108, the load device 110, the transceiver 112, etc., and may include various components that implement sensing, interface, communication, and convenience functions that are not directly involved in the driving operation.

When the vehicle 100 is driven based on electric energy, the energy generation unit 114 may be composed of, for example, an electric battery that is charged from the outside, or a combination of the electric battery and a fuel cell that charges the electric battery. When the vehicle 100 is driven based on fossil energy, the energy generation unit 114 may be composed of an internal combustion engine. When the vehicle 100 is a hybrid type, the energy generation unit 114 may be provided as a combination of the internal combustion engine and the electric battery.

The actuating unit 116 has at least one module for implementing a driving motion and may perform at least one driving motion among longitudinal control such as acceleration/deceleration and lateral control such as steering, according to a user request from the operating unit 106. The actuating unit 116 may have a wheel drive unit 118, a mechanical component for implementing the driving motion in the wheel drive unit 118, and/or an electronic module for executing a driving motion according to a command of the processor 122 by the manual operation of the user or the autonomous driving. When the vehicle 100 operates based on electric energy, the vehicle may include an assembly for transmitting the requested driving motion to the wheel drive unit 118. When the vehicle 100 operates based on fossil energy, the actuating unit 116 may include a transmission and a gear module for transmitting the power of the internal combustion engine.

The wheel drive unit 118 may include a motor that generates a plurality of wheel driving forces and applies the generated driving forces to the wheels, decelerates the driving of the wheels, and controls the lateral direction of the wheels.

The memory 120 may store at least one program (e.g., an operating system, software, firmware, middleware, multiple applications, etc.) for controlling the vehicle 100, various data, and at least one instruction, and may load a program, read or write data, or perform an operation corresponding to the instruction by a request of the processor 122. The memory 120 may include a volatile memory or a nonvolatile memory.

The processor 122 may perform overall control of the vehicle 100 according to an input command. The command may be input to the processor 122 by the memory 120 or the transceiver 112. For example, the processor 122 may execute the program or instruction stored in the memory 120 to control operations of other components (hardware or software) connected to the vehicle 100 and perform data processing and calculation.

The processor 122 may include, for example, at least one central processing unit (CPU), at least one microprocessor, and/or at least one digital signal processor (DSP). In addition, the processor 122 may load a command or data received from other components (e.g., the sensor unit 102 or the transceiver 112) into the volatile memory, process the command or data stored in the volatile memory, and store the processing result in the non-volatile memory.

Meanwhile, the vehicle 100 may include at least one vehicle controller. The at least one vehicle controller may be provided in the form of an embedded system inside the vehicle 100, and when a plurality of vehicle controllers are provided, they may be implemented as independent devices according to the functions of the vehicle controllers or may be connected to each other to be able to communicate with each other. In addition, the at least one vehicle controller may be implemented integrally with the vehicle internal control units (e.g., the processor 122) and/or implemented as a separate independent chip. For example, at least one controller may be implemented in various forms such as an electronic control unit (ECU), a micro controller unit (MCU), a CPU, a microprocessor, etc.

The function that the at least one vehicle controller may control may be one of various vehicle control functions including engine control, transmission control, electronic stability control, airbag control, a tire pressure monitoring system, motor control, seat control, door control, etc.

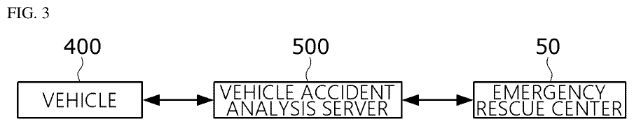

FIG. 3 is a diagram illustrating a vehicle accident analysis system according to an example of the present disclosure.

Referring to FIG. 3, the vehicle accident analysis system according to an example of the present disclosure may include a vehicle 400 and/or a vehicle accident analysis server 500.

The vehicle 400 is a vehicle equipped with an emergency call (eCall) system, and when a collision accident occurs, the vehicle may collect initial information necessary for accident analysis (hereinafter referred to as “initial accident information”) and transmit the collected initial information to the vehicle accident analysis server 500.

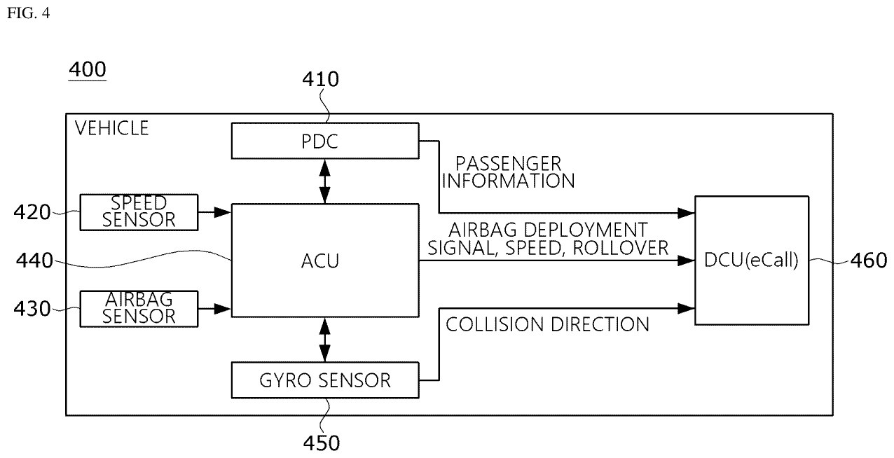

FIG. 4 is a block diagram illustrating the vehicle 400 according to an example of the present disclosure.

Referring to FIG. 4, the vehicle 400 may include a power-net domain controller (PDC) 410, a speed sensor 420, an airbag sensor 430, an airbag control unit (ACU) 440, a gyro sensor 450, and a data connectivity unit (DCU) 460. The PDC 410, the speed sensor 420, the airbag sensor 430, the ACU 440, the gyro sensor 450, and the DCU 460 may use controller area network (CAN) communication and local interconnect network (LIN) communication.

The PDC 410 may be the ECU that detects whether a seat belt is fastened and acquires and stores information on a passenger aboard the vehicle 400. The passenger information may include the number of passengers, seating positions of passengers, and whether each passenger is wearing a seat belt. The PDC 410 may detect whether the seat belt is fastened, generate and store the passenger information from the detected result, and transmit the passenger information to the DCU 460 when the airbag deployment signal is received from the ACU 440.

The speed sensor 420 may periodically detect the speed of the driving vehicle 400 and transmit the detected speed to the ACU 440. The speed sensor 420 may be a sensor installed in an engine management system (EMS), for example. The speed sensor 420 may detect the vehicle speed, for example, in units of 10 ms and transmit the detected vehicle speed to the ACU 440.

The airbag sensor 430 is installed at a location where the airbag module is installed in the vehicle 400 to detect whether to deploy the airbag, and when the airbag is deployed, transmit the airbag deployment signal to the ACU 440. For example, when N airbag modules are installed in vehicle 400, N airbag sensors 430 are also installed, and when some of the N airbag modules are deployed, the corresponding airbag sensors may transmit airbag deployment signals to some of the airbag modules.

The ACU 440 may be the ECU that monitors the collision situation of the vehicle 400 and determines whether to deploy the airbag. The ACU 440 may receive the sensing information from the plurality of collision sensors of the vehicle 400, determine whether to deploy the airbag, and transmit an airbag deployment command to the airbag module. The collision sensor may measure the collision intensity or collision direction and transmit the measured collision intensity or direction to the ACU 440.

In addition, the ACU 440 may periodically receive and store the vehicle speed from the speed sensor 420.

In addition, when the ACU 440 receives the airbag deployment signal from the airbag sensor 430, it may determine whether the vehicle 400 rolls over from the sensing value (e.g., Z-axis value) of the gyro sensor mounted on the ACU 440 and transmit the result on whether the vehicle rolls over to the DCU 460.

In addition, when the ACU 440 receives the airbag deployment signal, the ACU 440 may collect speeds periodically detected and stored for a set time (e.g., 250 ms) from the time when the airbag deployment signal is received from the speed sensor 420 (i.e., the time of the collision when the accident has occurred, which may be set to 0 ms) and may transmit the collected speed to the DCU 460. 250 ms is an example, but is not limited thereto, and may be increased or decreased.

In addition, when the ACU 440 receives the airbag deployment signal, the ACU may notify the PDC 410 and the gyro sensor 450 that the airbag deployment signal has been received.

The gyro sensor 450 may detect the collision direction information of the vehicle 400 at the time when the airbag deployment signal is received and transmit the detected collision direction information to the DCU 460. The collision direction information of the vehicle 400 may be used to determine the collision direction as the X-axis value and the Y-axis value of the vehicle 400. FIG. 5 is a diagram illustrating the X-axis, Y-axis, and Z-axis of the vehicle 400. The gyro sensor 450 may be mounted on a navigation system of the head unit.

The collision direction information of the vehicle 400 may also be detected using the acceleration sensor of the ACU 440 instead of the gyro sensor 450.

Therefore, when the airbag deployment signal is received, the PDC 410 transmits the most recently stored passenger information to the DCU 460, the ACU 440 transmits, to the DCU 460, the airbag deployment signal, the vehicle speeds periodically detected from the time when the airbag deployment signal is received for a certain period of time (e.g., 0 ms to 250 ms), and the result on whether the vehicle rolls over, and the gyro sensor 450 may transmit the X-axis value and Y-axis value of the vehicle 400 to the DCU 460. The vehicle speed detected at 0 ms may be considered the speed at the time of the collision, and the vehicle speed detected at 250 ms may be considered the final speed after the collision.

The DCU 460 may be a modem having an eCall function. The DCU 460 may generate initial accident information by collecting the passenger information, the vehicle speeds periodically detected for a certain period of time from the time of collision, the result on whether the vehicle rolls over, the X-axis value and Y-axis value of vehicle 400, and the vehicle identification number together with the airbag deployment signal, and transmit the generated initial accident information to the vehicle accident analysis server 500. The vehicle identification number of the vehicle 400 may be stored in the memory of the DCU 460. When the vehicle 400 uses the CCS, the DCU 460 may transmit the vehicle identification number, and when the vehicle 400 does not use the CCS, it may transmit attribute information of the vehicle 400, such as a vehicle weight and a vehicle type, by adding the attribute information to the initial accident information.

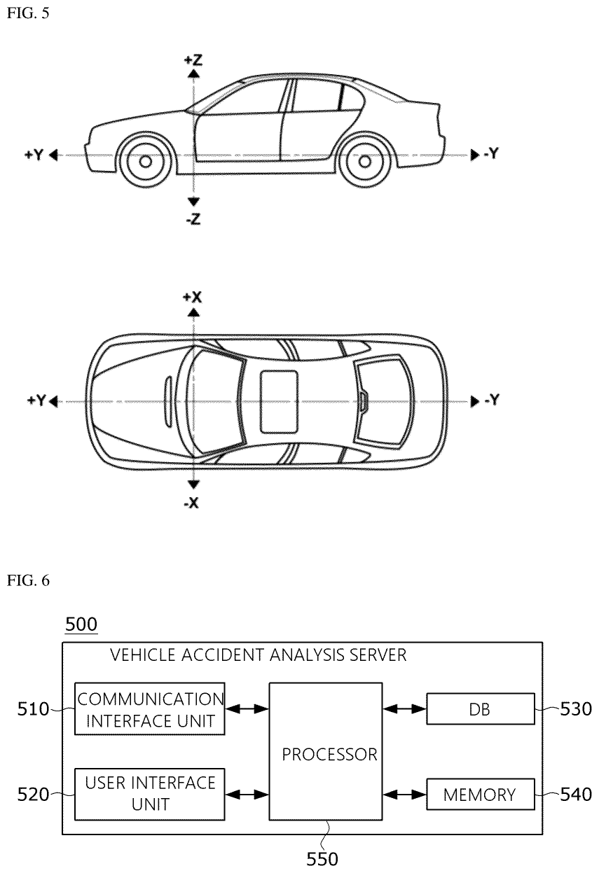

FIG. 6 is a block diagram illustrating the vehicle accident analysis server 500 according to an example of the present disclosure.

The vehicle accident analysis server 500 illustrated in FIG. 6 may analyze information required for accident analysis received from the vehicle 400 using a deep learning model, calculates the vehicle collision severity index (CSI) and injury severity of each passenger, and transmits the calculated results to the emergency rescue center 50 to share the calculated results with the emergency rescue center 50. The vehicle 400 may use the CCS, and the server 500 may be a server providing the CCS but is not limited thereto.

Referring to FIG. 6, the vehicle accident analysis server 500 according to an example of the present disclosure may include a communication interface unit 510, a user interface unit 520, a database (DB) 530, a memory 540, and a processor 550.

The communication interface unit 510 may communicate with the vehicle 400 and the emergency rescue center 50 through a network (not illustrated) via wired or wireless means. For example, the communication interface unit 510 may receive the initial accident information from the vehicle 400 and transmit the injury severity, the accident risk level, and the CSI for each passenger to the emergency rescue center 50.

The user interface unit 520 provides an interfacing path between the user and the vehicle accident analysis server 500. The user interface unit 520 may transmit the command input from the user to the processor 550, receive a processing result according to the command from the processor 550, and display the received processing result on the screen. The user interface unit 520 may include input devices such as a mouse, a keyboard, and a touch panel, and output devices such as a speaker and a display panel.

The DB 530 may store the attribute information related to the vehicle 400 based on the vehicle identification number of the vehicle 400. The attribute information may be diverse, and may store information such as the weight, the vehicle type, and the production year, etc., of the vehicle 400.

The memory 540 may store at least one program (e.g., an operating system, software, firmware, middleware, or an application, etc.), various data, and/or at least one command to implement or provide an operation or function provided by the vehicle accident analysis server 500, and loads programs, reads or records data, or performs an operation corresponding to the command at the request of the processor 550. The memory 170 may include a volatile memory or a nonvolatile memory.

The program stored in the memory 540 may include a vehicle accident analysis program. The vehicle accident analysis program may be implemented as a deep learning-based artificial intelligence model including multiple codes or instructions that may analyze the initial accident information received from vehicle 400 to generate the information related to the accident of the vehicle 400 (hereinafter referred to as “vehicle accident information”), and analyze the vehicle accident information to calculate or estimate the CSI, the accident risk level, and the injury severity of each passenger of the vehicle 400.

In addition, the vehicle accident analysis program may include airbag coefficients and values such as in [Table 1] to [Table 4].

[Table 1] shows an example of a collision energy coefficient k1, a speed coefficient k2, and a vehicle weight coefficient k3 for each vehicle type.

TABLE 1

| Vehicle Type | k1 | k2 | k3 |
|---|---|---|---|
| Compact Car | 0.0004 | 0.05 | 0.005 |
| Sedan | 0.0006 | 0.07 | 0.007 |
| SUV | 0.0008 | 0.09 | 0.010 |

k1, k2, and k3 may be further subdivided according to the vehicle weight.

[Table 2] shows an example of direction coefficients mapped to the vehicle collision angle or the vehicle collision direction.

TABLE 2

| Collision | Direction |
|---|---|
| Direction | Coefficient |
| Frontal Collision | 1.2 |
| Oblique Collision | 1.1 |
| Rear-end Collision | 0.9 |
| Lateral Collision | 0.8 |

Referring to [Table 2], a frontal collision is generally the most dangerous because the speed of the vehicle 400 decreases the most, and passengers move forward significantly and are likely to be injured. A lateral collision may be less dangerous than a frontal collision because the side of the vehicle 400 has doors, side airbags, and shock absorbing structures. A rear-end collision is considered to be relatively less dangerous than frontal or lateral collisions. The reason is that there is usually more space at the rear, and there is a distance from the location where the passenger is on board. In addition, the vehicle speed often does not decrease rapidly in the case of a rear-end collision, so the shock may be less severe.

[Table 3] shows the accident risk level mapped to the CSI.

TABLE 3

| | Accident |
|---|---|
| CSI | Risk Level |
| CSI ≥ 400 | 3 (Severe) |
| 200 < CSI < 400 | 2 (Moderate) |
| CSI ≤ 200 | 1 (Minor) |

200 and 400 in [Table 3] are examples and can be changed, and the accident risk level may be subdivided into 4 or more.

[Table 4] shows examples of weights for each seat in the vehicle.

TABLE 4

| Seat Location | Seat Weight |
|---|---|
| Driver's Seat | 1.2 |
| Passenger's Seat | 1.1 |
| Rear Left Seat | 1.05 |
| Rear Center Seat | 1.0 |
| Rear Right Seat | 1.05 |

The processor 550 controls the overall operation of the vehicle accident analysis server 500 by executing one or more operating systems or programs stored in the memory 540. The processor 550 may include, for example, a CPU, a graphics processing unit (GPU), a MCU, an application processor (AP), or at least one electronic device capable of performing various operations and control processing. These devices may be implemented by using, for example, one or more semiconductor chips, circuits, or related components, alone or in combination.

In an example of the present disclosure, the processor 550 may determine the accident risk level of the accident vehicle 400 based on the vehicle accident information and estimate the injury severity of each passenger in the accident vehicle based on the determined accident risk level. In addition, the processor 550 may transmit the injury severity of each passenger to the emergency rescue center 50 or process the injury severity so that it is displayed on the user interface unit 520.

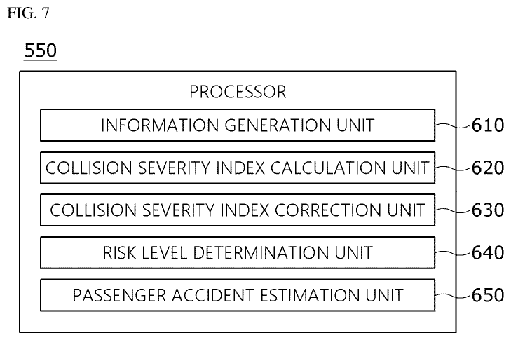

FIG. 7 is a block diagram illustrating a configuration of the processor 550 according to an example of the present disclosure.

Referring to FIG. 7, the processor 550 may include an information generation unit 610, a CSI calculation unit 620, a CSI correction unit 630, a risk level determination unit 640, and a passenger accident estimation unit 650. Components 610 to 650 represent functionally distinct elements, and two or more elements may be implemented by physically integrating each other, or each element may be implemented in a physically distinct form. In addition, at least one of the components 610 to 650 may be implemented in the form of the command stored in the memory 540.

The information generation unit 610 may process the initial accident information received from the DCU 460 to generate the vehicle accident information necessary for actually analyzing the accident of the vehicle 400. The initial accident information may include the airbag deployment signal indicating that the airbag in the accident vehicle 400 has been deployed, the passenger information, the vehicle speeds periodically detected for a certain period of time from the time of the collision, the result on whether the vehicle rolls over, the X-axis and Y-axis values of the vehicle 400 which are the gyro sensing values, and the vehicle identification number.

For example, the information generation unit 610 may search for the weight and vehicle type of the vehicle 400 mapped to the vehicle identification number among the initial accident information from the DB 530.

In addition, the information generation unit 610 may calculate the vehicle speed change amount from the initial vehicle speed (the speed detected at 0 ms, which is referred to as the “speed at the time of the collision”) among the initial accident information and the vehicle speed detected after a certain period of time (the speed detected at 250 ms, which is referred to as the “final speed after the collision”). The vehicle speed change amount may be directly calculated by the ACU 440 and transmitted to the server 500.

In addition, the information generation unit 610 may calculate the collision angle of the vehicle 400 from the X-axis value and Y-axis value of the vehicle 400, and determine the collision direction from the calculated collision angle. The information generation unit 610 may calculate the vehicle collision angle using, for example, a math.a tan 2 function. The collision direction may include a frontal collision, an oblique collision, a rear-end collision, and a lateral collision. For example, when the calculated collision angle is within a range of 315° to 45°, the information generation unit 610 may determine that a frontal collision has occurred.

The information generation unit 610 may generate vehicle accident information by collecting the airbag deployment signal, the passenger information, the vehicle speed change amount, the final speed after the collision, the result on whether the vehicle rolls over, the collision angle, the vehicle weight, and the vehicle type.

The CSI calculation unit 620 may calculate the CSI based on a plurality of pieces of first information among the generated vehicle accident information. The CSI is an index that numerically represents the CSI by considering the energy, speed, deformation, etc., of the vehicle when an accident occurs. Among the vehicle accident information, the plurality of pieces of first information may include the weight of the accident vehicle 400, the vehicle speed change amount due to the accident, and the final speed after the collision.

The CSI calculation unit 620 may calculate the CSI using the following [Equation 1].

$$CSI = k 1 \times e + k 2 \times {( {V}_{final} )}^{2} + k 3 \times M \qquad [ Equation 1 ]$$

In the above [Equation 1], k1 denotes a collision energy coefficient, e denotes a collision energy (J), k2 denotes a speed coefficient, V_{final} denotes a final speed of the vehicle 400 after the collision (m/s), k3 denotes a vehicle weight coefficient, and M denotes a vehicle weight (kg). e=0.5×M×V². The larger the CSI, the more serious the accident, and the lower the CSI, the more minor the accident.

The CSI correction unit 630 may correct the CSI calculated by the CSI calculation unit 620 based on a plurality of pieces of second information among the vehicle accident information. The plurality of pieces of second information among the vehicle accident information may include the airbag deployment signal and the vehicle collision angle.

The CSI correction unit 630 may primarily correct the CSI by reducing the CSI using the airbag coefficient mapped to the airbag deployment signal. In general, when the airbag is deployed, the injury severity is reduced by about 30%. Therefore, the CSI correction unit 630 may primarily correct the CSI by multiplying the CSI calculated by the CSI calculation unit 620 by an airbag coefficient of 0.7. For example, 30%, 0.7, etc., can be changed by an administrator of the server 500.

In addition, the CSI correction unit 630 may confirm the direction coefficient mapped to the vehicle collision angle, i.e., the vehicle collision direction, from the DB 530, and additionally correct the primarily corrected CSI with the confirmed direction coefficient to calculate the final CSI. For example, when the collision direction of the vehicle 400 is a frontal collision, the CSI correction unit 630 may additionally correct the CSI by multiplying the primarily corrected CSI by a direction coefficient of 1.2.

The risk level determination unit 640 may determine the accident risk level of the accident vehicle 400 based on the final CSI calculated by the CSI correction unit 630. Referring to [Table 3], the risk level determination unit 640 may determine the accident risk level as 1 when the CSI is 200 or less, and 3 when the accident risk level is 400 or more.

The passenger accident estimation unit 650 may estimate the injury severity of each passenger in the accident vehicle 400 based on the accident risk level determined by the risk level determination unit 640 and the plurality of pieces of third information among the vehicle accident information. The plurality of pieces of third information are the information on the passengers aboard the accident vehicle 400, and may include the number of passengers, the passengers' boarding seat locations, and whether the passengers are wearing seat belts.

The passenger accident estimation unit 650 may temporarily calculate the injury severity of each passenger using the seat weights set for each boarding seat location and the determined accident risk level. For example, when a driver is on board and when the seat weight mapped to the driver's seat is 1.2, and the accident risk level is 3, the passenger accident estimation unit 650 may temporarily calculate the injury severity of the driver's seat as 3.6 by multiplying the driver's weight of 1.2 by 3.

In addition, the passenger accident estimation unit 650 may correct the temporarily calculated injury severity of each passenger using whether the passenger is wearing a seat belt, and determine the corrected result as the final injury severity of each passenger. When the passenger is not wearing a seat belt, the passenger accident estimation unit 650 may calculate the final injury severity by multiplying the temporarily calculated injury severity by the belt weight. For example, when the driver is not wearing a seat belt, the passenger accident estimation unit 650 calculates the final injury severity by multiplying the temporarily calculated driver's injury severity of 3.6 by the belt weight of 1.5.

The passenger accident estimation unit 650 may control the communication interface unit 510 to confirm the final injury severity or the injury type mapped to the final injury severity and to transmit the confirmed injury type to the emergency rescue center 50 together with the attribute information of the vehicle 400. The injury type may be, for example, minor injury, moderate injury, or serious injury.

The emergency rescue center 50 may more accurately predict the passenger condition and make an early determination on the rescue or treatment method based on the final injury severity or injury type estimated for each passenger.

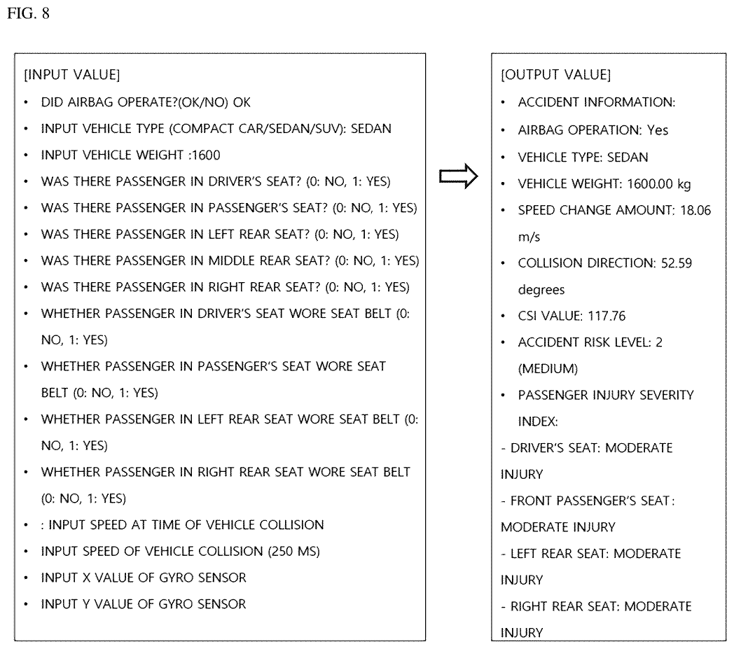

FIG. 8 is a diagram illustrating an example of the initial accident information input to the vehicle accident analysis server 500 and the output value generated from the initial accident information.

In the example depicted in FIG. 8, according to the initial accident information that is the input value, the vehicle type is a sedan, the weight is 1600 kg, the number of passengers is 4, and only the driver is wearing a seat belt. In addition, in that example, at the time of the vehicle collision or when the airbag deployment signal is input, the speed is 75 km/h, and the final vehicle speed, i.e., the speed after the vehicle collision, is 10 km/h. The output values generated from the initial accident information include whether the airbag is deployed, the vehicle type, the vehicle weight, the speed change amount, the collision direction, the CSI value, the accident risk level, and the injury severity of each passenger. The input value includes the vehicle identification number, and the vehicle type and weight indicated in the input value may be included in the output value.

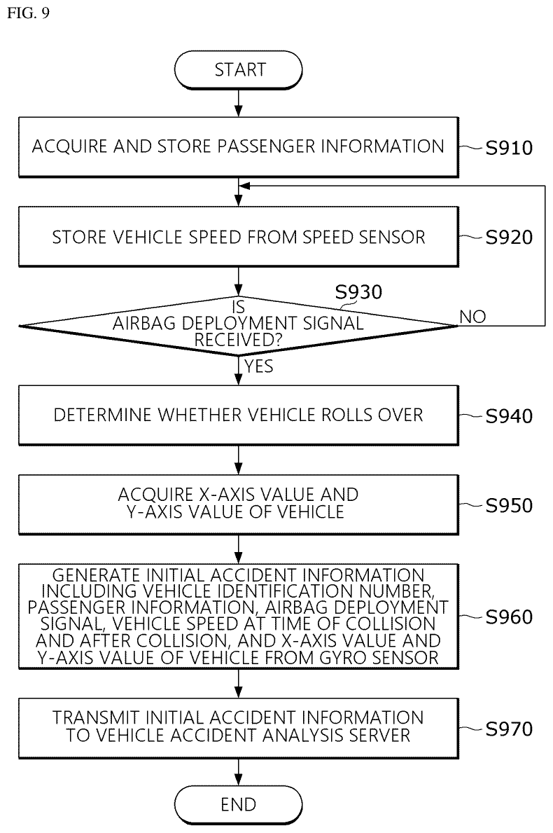

FIG. 9 is a flowchart illustrating a method of providing initial accident information of the vehicle 400 according to an example of the present disclosure.

Referring to FIG. 9, the PDC 410 of the vehicle 400 can obtain and store the information on the passengers aboard the vehicle 400 (S910). The passenger information includes the number of passengers, the boarding positions, and whether the passengers are wearing seat belts.

The ACU 440 stores the speeds of the vehicle 400 periodically detected by the speed sensor 420 (S920).

When the airbag deployment signal is received from the airbag sensor 430 (S930—Yes), the ACU 440 determines whether the vehicle 400 rolls over (S940), and the gyro sensor 450 may detect the X-axis value and Y-axis value of the vehicle 400 at the time when the airbag deployment signal is received or occurs (S950).

The DCU 460 generates initial accident information including the passenger information, the speed at the time of the collision and the speed after the collision of the vehicle 400, the airbag deployment signal, whether the vehicle rolls over, the X-axis value and Y-axis value of the vehicle 400, and/or the vehicle identification number (S960).

The DCU 460 may transmit the generated initial accident information to the vehicle accident analysis server 500 (S970).

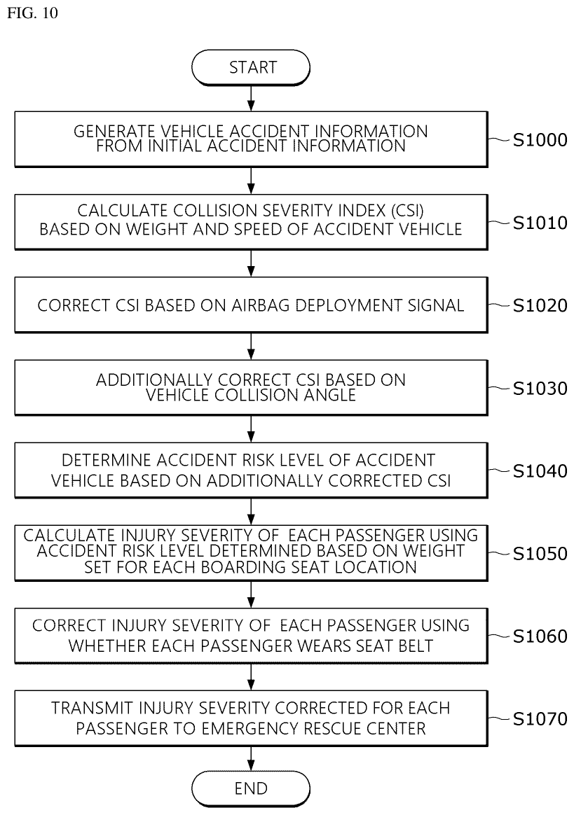

FIG. 10 is a flowchart illustrating a vehicle accident analysis method according to an example of the present disclosure.

Referring to FIG. 10, the information generation unit 610 of the vehicle accident analysis server 500 processes the initial accident information received from the accident vehicle 400 and generates the vehicle accident information necessary for the vehicle accident analysis (S1000).

The CSI calculation unit 620 may calculate the CSI using the vehicle weight, the speed at the time of the collision and the speed after the collision of the vehicle 400, and the above [Equation 1] among the vehicle accident information (S1010).

The CSI correction unit 630 may primarily correct the CSI calculated in operation S1010 based on the airbag deployment signal among the vehicle accident information (S1020). Operation S1020 may reduce the CSI by multiplying the airbag coefficient mapped to the airbag deployment signal by the CSI.

In addition, the CSI correction unit 630 may further correct the primarily added CSI based on the vehicle collision angle, i.e., the collision direction among the vehicle accident information (S1030). In operation S1030, the CSI is additionally corrected by multiplying the primarily corrected CSI by the direction coefficient mapped to the collision direction.

The risk level determination unit 640 determines the accident risk level of the accident vehicle 400 based on the CSI additionally corrected in operation S1030 (S1040).

The passenger accident estimation unit 650 temporarily calculates the injury severity of each passenger based on the accident risk level determined in operation S1040 and the passenger location among the vehicle accident information (S1050). In operation S1050, the injury severity of each passenger may be temporarily calculated by multiplying the seat weight mapped to the passenger's seat location by the accident risk level.

The passenger accident estimation unit 650 may calculate the final injury severity by correcting the temporarily calculated injury severity of each passenger using whether the passenger is wearing a seat belt (S1060). In operation S1060, for the passenger wearing the seat belt, the correction may be made by multiplying the injury severity of the passenger calculated in operation S1050 by the belt weight.

The passenger accident estimation unit 650 may transmit the final injury severity calculated for each passenger to the emergency rescue center 50.

In an example, a server (e.g., a vehicle accident analysis server) may communicate with a vehicle and may monitor accidents associated with the vehicle. The server may include one or more processors and a memory configured to store one or more programs executed by the one or more processors. The one or more processors are configured to cause the vehicle accident analysis server to receive, from an accident vehicle via a communication interface, vehicle accident information, wherein the vehicle accident information indicates accidents associated with the accident vehicle, determine an accident risk level of the accident vehicle based on the vehicle accident information indicating information related to an accident of the accident vehicle, estimate injury severity of each passenger in the accident vehicle based on the determined accident risk level, and transmit, based on the estimated injury severity, a control signal to the accident vehicle to cause the accident vehicle to adjust at least one parameter for autonomous driving control of the accident vehicle.

In an example, a computing device may be located in a vehicle and may be configured to monitor and report accidents associated with the vehicle. The computing device may detect, via one or more sensors, an occurrence of an accident associated with the vehicle. The computing device may then calculate, based on data from a gyro of the vehicle, a vehicle collision angle and calculate, based on the vehicle collision angle and one or more properties of the vehicle, a collision severity index corresponding to the accident. Then, the computing device may obtain, based on seat belt sensors of the vehicle, passenger data corresponding to one or more passengers in the vehicle at a time of the accident and estimate, based on the collision severity index, an injury level for each of the one or more passengers. The computing device may then transmit, to a remote server, the estimated injury level for each of the one or more passengers.

Exemplary methods of the present disclosure described above are expressed as a series of operations for clarity of explanation, but this is not intended to limit the order in which steps are performed, and the steps may be performed simultaneously or in a different order, if necessary. In order to implement the method according to the present disclosure, other steps may be included in addition to the exemplified steps, some steps may be omitted and the others included, or some steps may be omitted and other additional steps included.

Various examples of the present disclosure are intended to explain representative aspects of the present disclosure, rather than listing all possible combinations, and matters described in various examples may be applied independently or in a combination of two or more.

In addition, various examples of the present disclosure may be implemented by hardware, firmware, software, a combination thereof, or the like. For implementation by hardware, various examples of the present disclosure may be implemented by one or more application specific integrated circuits (ASICs), DSPs, digital signal processing devices (DSPDs), programmable logic devices (PLDs), field programmable gate arrays (FPGAs), processors, controllers, microcontrollers, microprocessors, or the like.

The scope of the present disclosure includes software or machine-executable instructions (e.g., operating systems, applications, firmware, programs, etc.) that cause operations according to the methods of various examples to be executed on a device or computer, and a non-transitory computer-readable medium in which such software, instructions, etc., are stored and executable on a device or computer.

The existing emergency rescue services or eCall services are passive activities as they can only determine whether an accident has occurred, the vehicle location, and whether an airbag has been deployed. However, according to the present disclosure, it is possible to provide more accurate information using the deep learning system that estimates the injury severity.

In addition, according to the present disclosure, when an accident occurs in a vehicle, collision direction data is secured, and the degree of damage to each passenger is estimated using the secured collision direction data and then transmitted to the emergency center, thereby helping to identify the passenger condition before rescue.

In addition, according to the present disclosure, by sensing and providing the collision angle or collision location of the accident vehicle, it may be possible to perform the eCall+ authentication response and increase the accuracy of the accident severity, the accident risk level, and the injury severity of each passenger.

In addition, according to the present disclosure, since the vehicle may operate on the server's software logic, it is possible to continuously supplement and update the software by reflecting new information such as additional information, accident cases, and analysis information obtained during vehicle development as vehicles are further developed in the future.

In addition, according to the present disclosure, different countries that provide CCSs may have separate dedicated servers, and the present disclosure can be implemented based on the CAN signal of the vehicle, and therefore can be applied both domestically and internationally.

In addition, according to the present disclosure, in the case of a region where a server for CCSs is not operated and only the eCall function is applied, the logic of the present disclosure can be applied to a DCU/HU/third unit, etc., to transmit the information to the emergency rescue center of the country.

What is claimed is:

1. A vehicle accident analysis server comprising:
one or more processors; and
a memory configured to store one or more programs executed by the one or more processors,
wherein the one or more processors are configured to cause the vehicle accident analysis server to:
receive, from an accident vehicle via a communication interface, vehicle accident information, wherein the vehicle accident information indicates accidents associated with the accident vehicle,
determine an accident risk level of the accident vehicle based on the vehicle accident information indicating information related to an accident of the accident vehicle,
estimate injury severity of each passenger in the accident vehicle based on the determined accident risk level, and
transmit, based on the estimated injury severity, a control signal to the accident vehicle to cause the accident vehicle to adjust at least one parameter for autonomous driving control of the accident vehicle.

2. The vehicle accident analysis server of claim 1, wherein the one or more processors are further configured to cause the vehicle accident analysis server to:
calculate a collision severity index (CSI) based on a plurality of pieces of first information among the vehicle accident information;
correct the calculated CSI based on a plurality of pieces of second information among the vehicle accident information; and
determine the accident risk level of the accident vehicle based on the corrected CSI.

3. The vehicle accident analysis server of claim 2, further comprising:
the communication interface that:
receives a vehicle identification number from the accident vehicle and vehicle speeds periodically measured for a set time from a time when the accident occurs,
wherein the one or more processors are further configured to cause the vehicle accident analysis server to:
confirm a vehicle weight mapped to the received vehicle identification number in a database (DB) to use the confirmed vehicle weight as a weight of the accident vehicle, and
calculate a vehicle speed change amount from the periodically measured vehicle speeds,
wherein the plurality of pieces of first information among the vehicle accident information include the weight of the accident vehicle, the vehicle speed change amount, and a vehicle speed at a time when a set time has elapsed from the time when the accident occurs.

4. The vehicle accident analysis server of claim 2, wherein the plurality of pieces of second information include an airbag deployment signal indicating that an airbag in the accident vehicle has been deployed, and
wherein the one or more processors are configured to cause the vehicle accident analysis server to reduce the calculated CSI using a coefficient mapped to the airbag deployment signal to perform a primary correction.

5. The vehicle accident analysis server of claim 4, wherein the plurality of pieces of second information further include a vehicle collision angle calculated from a gyro sensing value of the accident vehicle, and
wherein the one or more processors are configured to cause the vehicle accident analysis server to correct the CSI using a coefficient mapped to the calculated vehicle collision angle.

6. The vehicle accident analysis server of claim 5, wherein the accident vehicle transmits an X-axis value and a Y-axis value of a gyro sensor detected at a time when the airbag deployment signal is input, and
wherein the one or more processors are further configured to cause the vehicle accident analysis server to calculate the vehicle collision angle based on the X-axis value and Y-axis value of the gyro sensor.

7. The vehicle accident analysis server of claim 5, wherein the one or more processors are further configured to cause the vehicle accident analysis server to:
classify a collision type of the accident vehicle as one of frontal collision, oblique collision, rear-end collision, and lateral collision based on the calculated vehicle collision angle, and
correct the primarily corrected CSI using a coefficient mapped to the classified collision type.

8. The vehicle accident analysis server of claim 1, wherein the one or more processors are further configured to cause the vehicle accident analysis server to:
estimate the injury severity of each passenger based on the accident risk level determined based on a plurality of pieces of third information among the vehicle accident information.

9. The vehicle accident analysis server of claim 8,
wherein the plurality of pieces of third information are information on passengers aboard the accident vehicle, and include a number of passengers, boarding seat locations of the passengers, and whether the passengers are wearing seat belts, and
wherein the one or more processors are further configured to cause the vehicle accident analysis server to calculate the injury severity of each passenger using a weight set for each passenger seat location and the determined accident risk level.

10. The vehicle accident analysis server of claim 9, wherein the one or more processors are further configured to cause the vehicle accident analysis server to correct the injury severity calculated for each passenger based on whether each passenger is wearing a seat belt and determine the corrected injury severity as final injury severity of each passenger.

11. A vehicle accident analysis method performed by a server including a processor and a memory configured to store one or more programs executed by the processor, the method comprising:
receiving, from an accident vehicle via a communication interface, vehicle accident information, wherein the vehicle accident information indicates accidents associated with the accident vehicle;
determining an accident risk level of the accident vehicle based on the vehicle accident information indicating information related to an accident of the accident vehicle;
estimating injury severity of each passenger in the accident vehicle based on the determined accident risk level; and
transmitting, based on the estimated injury severity, a control signal to the accident vehicle to cause the accident vehicle to adjust at least one parameter for autonomous driving control of the accident vehicle.

12. The vehicle accident analysis method of claim 11, wherein the determining of the accident risk level includes:
calculating a collision severity index (CSI) based on a plurality of pieces of first information among the vehicle accident information;
correcting the calculated CSI based on a plurality of pieces of second information among the vehicle accident information; and
determining the accident risk level of the accident vehicle based on the corrected CSI.

13. The vehicle accident analysis method of claim 12, further comprising:
prior to the calculating of the CSI:
confirming a vehicle weight mapped to a vehicle identification number in a database (DB) to use the confirmed vehicle weight as a weight of the accident vehicle, and
calculating a vehicle speed change amount from periodically measured vehicle speeds,
wherein the server is configured to receive, from the accident vehicle, the vehicle identification number and the vehicle speeds periodically measured for a set time from a time when the accident occurs.

14. The vehicle accident analysis method of claim 13, wherein the plurality of pieces of first information among the vehicle accident information include the weight of the accident vehicle, the vehicle speed change amount, and a vehicle speed at a time when a set time has elapsed from the time when the accident occurs.

15. The vehicle accident analysis method of claim 12,
wherein the plurality of pieces of second information include an airbag deployment signal indicating that an airbag in the accident vehicle has been deployed, and
wherein the correcting of the CSI comprises reducing the calculated CSI using a coefficient mapped to the airbag deployment signal to perform a primary correction.

16. The vehicle accident analysis method of claim 15,
wherein the plurality of pieces of second information further include a vehicle collision angle calculated from a gyro sensing value of the accident vehicle, and
wherein the correcting of the CSI comprises correcting the CSI using a coefficient mapped to the calculated vehicle collision angle.

17. The vehicle accident analysis method of claim 16, further comprising, prior to the correcting of the calculated CSI, calculating the vehicle collision angle based on an X-axis value and a Y-axis value of a gyro sensor,
wherein the accident vehicle transmits the X-axis value and Y-axis value of the gyro sensor detected at a time when the airbag deployment signal is input.

18. The vehicle accident analysis method of claim 16, wherein the correcting of the calculated CSI comprises:
classifying a collision type of the accident vehicle as one of frontal collision, oblique collision, rear-end collision, and lateral collision based on the calculated vehicle collision angle, and
correcting the primarily corrected CSI using a coefficient mapped to the classified collision type.

19. The vehicle accident analysis method of claim 11, wherein the estimating of the injury severity comprises estimating the injury severity of each passenger based on the accident risk level determined in the determining of the risk level and a plurality of pieces of third information among the vehicle accident information.

20. A computing device in a vehicle and configured to monitor and report accidents associated with the vehicle, the computing device comprising:
one or more processors; and
memory storing instructions that, when executed by the one or more processors, cause the computing device to:
detect, via one or more sensors, an occurrence of an accident associated with the vehicle;
calculate, based on data from a gyro sensor of the vehicle, a vehicle collision angle;
calculate, based on the vehicle collision angle and one or more properties of the vehicle, a collision severity index corresponding to the accident;
obtain, based on seat belt sensors of the vehicle, passenger data corresponding to one or more passengers in the vehicle at a time of the accident;
estimate, based on the collision severity index, an injury level for each of the one or more passengers; and
transmit, to a remote server via a wireless communication interface, the estimated injury level for each of the one or more passengers.
